(19) United States
(12) Patent Application Publication
(10) Pub. No.: US 2006/0030056 A1
(43) Pub. Date: Feb. 9, 2006

(54) Use of magnetic material to fractionate samples

(75) Inventors: Thomas L. Fort, Hanover, PA (US); Matthew Paul Collis, Seven Valleys, PA (US)

Correspondence Address: DAVID W HIGHET VP AND CHIEF IP COUNSEL;BECTON DICKINSON AND COMPANY, 1 BECTON DRIVE, MC110, FRANKLIN LAKES, NJ, 07417-1880, US

(73) Assignee: Becton, Dickinson and Company, Franklin Lakes, NJ (US)

(21) Appl. No.: 11/195,657

(22) Filed: Aug. 3, 2005

Related U.S. Application Data
(60) Provisional application No. 60/598,117, filed on Aug. 3, 2004.

(51) Int. Cl. G01N 33/551

(52) U.S. Cl. 436/524

(57) ABSTRACT

A method useful for the reversible binding of a protein molecule in a biological sample. The method uses paramagnetic particles having an associated electronic charge to bind proteins with the opposite charge to form a particle/protein complex. The complex can be immobilized to a container wall by applying a magnetic field to the particle/protein complex. The sample may be further processed to obtain a protein sample in a more pure form or a sample depleted of select proteins.

The present application claims priority to U.S. patent application Ser. No. 60/598,117 filed Aug. 3, 2004, the entire contents of which are incorporated by reference herein.

### FIELD OF THE INVENTION

The present invention relates generally to a composition and a method useful for the reversible binding of protein. More particularly, the present invention relates to a paramagnetic compound useful for extracting proteins non-specifically from solution.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Historically, protein purification schemes have been predicated on differences in the molecular properties of size, charge and solubility between the protein to be purified and undesired protein contaminants. Protocols based on these parameters include size exclusion chromatography, ion exchange chromatography, differential precipitation and the like.

Size exclusion chromatography, otherwise known as gel filtration or gel permeation chromatography, relies on the penetration of macromolecules in a mobile phase into the pores of stationary phase particles. Differential penetration is a function of the hydrodynamic volume of the particles. Accordingly, under ideal conditions, the larger molecules are excluded from the interior of the particles, while the smaller molecules are accessible to this volume and the order of elution can be predicted by the size of the protein because a linear relationship exists between elution volume and the log of the molecular weight.

Ion exchange chromatography involves the interaction of charged functional groups in the sample with ionic functional groups of opposite charge on an adsorbent surface. Two general types of interaction are known. The first is anionic exchange chromatography mediated by negatively charged amino acid side chains (e.g. aspartic acid and glutamic acid) interacting with positively charged surfaces. The second is cationic exchange chromatography mediated by positively charged amino acid residues (e.g., lysine and arginine) interacting with negatively charged surfaces.

Precipitation methods are predicated on the fact that in crude mixtures of proteins the solubilities of individual proteins are likely to vary widely. Although the solubility of a protein in an aqueous medium depends on a variety of factors, for purposes of this discussion, it can be said generally that a protein will be soluble if its interaction with the solvent is stronger than its interaction with protein molecules of the same or similar kind. Without wishing to be bound by any particular mechanistic theory describing precipitation phenomena, it is nonetheless believed that the interaction between a protein and water molecules occurs by hydrogen bonding with several types of uncharged groups and electrostatically, as dipoles with charged groups, and that precipitants such as salts of monovalent cations (e.g., ammonium sulfate) compete with proteins for water molecules. Thus, at high salt concentrations, the proteins become “dehydrated,” thereby reducing their interaction with the aqueous environment and increasing the aggregation with like or similar proteins, resulting in precipitation from the medium. Precipitation techniques, however, are crude. They also have the disadvantage of requiring filtration or centrifugation followed by resuspension and dialysis or some other form of buffer exchange to reduce salt concentration prior to downstream manipulation.

More recently, affinity chromatography and hydrophobic interaction chromatography techniques have been developed to supplement the more traditional size exclusion and ion exchange chromatographic protocols. Affinity chromatography relies on the interaction of the protein with an immobilized ligand. The ligand can be specific for the particular protein of interest, in which case the ligand is a substrate, substrate analog, inhibitor or antibody. Alternatively, the ligand may be able to react with a number of proteins. Such general ligands as adenosine monophosphate, adenosine diphosphate, nicotine adenine dinucleotide or certain dyes may be employed to recover a particular class of proteins.

Metal affinity partitioning exploits the affinity of transition metal ions for electron-rich amino acid residues, such as histidine and cysteine, accessible on the surfaces of some proteins. When the metal ion is partially chelated and coupled to a linear polymer, such as polyethylene glycol (“PEG”), the resulting polymer-bound metal chelate can be used to enhance the partitioning of metal binding proteins into the polymer-rich phase of a PEG-salt or PEG-dextran aqueous two-phase system.

The application of a metal affinity ligand for the isolation of proteins is known. It has been demonstrated that histidine- and cysteine-containing proteins could be chromatographically separated from each other using a support that had been functionalized with a chelator, such as iminodiacetic acid (“IDA”), which is attached to a polymer spacer and bound to a metal such as copper, zinc or nickel. Immobilized metal affinity chromatography (“IMAC”) has evolved into a useful tool for protein chromatography and a number of IDA-based IMAC resins are now commercially available.

Many problems occur when using metal chelates to purify a target protein from a crude preparation. One problem in particular centers on the selectivity of the ligand for the target protein, i.e., the ligand can be under or over selective in binding the target protein. There also is a problem of nitrogen-containing compounds in a crude system inhibiting ligand binding to the target protein. Finally, there is a problem relating to protein solubility and potential precipitation of proteins by the salt used in an aqueous, two-phase partitioning system. All of these problems can dramatically affect the target protein yield.

U.S. Pat. No. 5,907,035 (Guinn) has addressed the problems associated with metal chelation by developing an aqueous, two-phase metal affinity partitioning system for purifying target proteins from crude protein solutions. The method includes the use of salts and inert hydrophobic molecules, such as polymers, to produce the aqueous two-phase system and the use of a polymer-chelator-metal complex to purify target proteins by selectively binding them to the complex.

Despite continuous advances in these separation techniques, an effective and automated method of rapidly fractionating protein from crude biological samples has not been available. Precipitation techniques are still crude and difficult to automate. Chromatography is expensive and time consuming. Thus, there remains a need for a technique to rapidly fractionate proteins in crude biological samples.

### SUMMARY OF THE INVENTION

It is an object of the present invention to provide a non-specific capture technology that does not require coatings or bound ligands.

It is a further object of the present invention to provide a means to fractionate a biological sample containing proteins.

It is yet another object of the present invention to provide a process for reversibly binding a protein.

It is another object of the present invention to use magnetism to extract from a solution a particle-protein complex of a charged protein and a charged paramagnetic particle that has a charge opposite that of the charged protein.

To provide a more effective and efficient technique for the purification and manipulation of proteins, the present invention relates to a composition useful for reversibly binding proteins or peptide molecules. The composition comprises a paramagnetic particle in an environment that promotes binding. In one aspect, the invention also comprises the composition packaged as a kit, as well as methods of utilizing this composition to reversibly bind a protein molecule or adduct thereof.

In another aspect, this invention provides a method for fractionating a protein sample. The fractionating method comprises adding a paramagnetic particle to a sample comprising one or more proteins, where the proteins have at least one associated electronic charge. The method further comprises associating an electronic charge with the paramagnetic particle, wherein the charge is opposite that of the protein electronic charge such that the paramagnetic particle and the protein can form a complex. The complex is immobilized by applying a magnetic field. The material not immobilized by the magnetic field can then be removed for further analysis or disposed of as waste. The magnetic field is then removed to release the complex. Once the complex is no longer immobilized, a wash solution can be added if desired. The wash solution should be of such a composition that the opposing charges of bound protein and particle remain in effect and other materials can be released or washed from the complex. Upon re-application of the magnetic field, the complex can be immobilized and the immobilized material can then be removed and disposed of as waste. The electronic charge on the paramagnetic particle can then be altered, allowing the paramagnetic particle and the protein to dissociate. The magnetic field can be re-applied to immobilize the paramagnetic particle to aid in extracting the now fractionated protein sample.

A method of the present invention may comprise a) adding at least one paramagnetic particle elected from the group consisting of iron oxide, iron sulfide, iron chloride, ferric hydroxide, and ferrosoferric oxide to said sample comprising one or more proteins, at least one of said proteins having a first electronic charge; b) associating a second electronic charge with said at least one paramagnetic particle, wherein said second electronic charge is opposite that of said first electronic charge such that said at least one paramagnetic particle and said proteins are capable of forming a protein-particle complex; c) immobilizing said complex by applying a first magnetic field; d) removing material from said sample that is not immobilized by said first magnetic field; e) removing said first magnetic field from the remaining material to release said immobilized complex; f) altering said second electronic charge on said at least one paramagnetic particle, such that said complex disassociates; g) applying a second magnetic field to immobilize said at least one paramagnetic particle; and h) extracting said protein from remaining material.

A method of the invention may also comprise a method as described above wherein said at least one paramagnetic particle is a metal compound selected from the group consisting of an iron compound, a cobalt compound, and a nickel compound.

A method of the invention may also comprise a method as described above wherein said iron compound selected from the group consisting of iron oxide, iron sulfide, iron chloride, ferric hydroxide, and ferrosoferric oxide.

A method of the invention may also comprise a method as described above wherein an acid is used to associate said second electronic charge with said paramagnetic particle.

A method of the invention may also comprise a method as described above wherein said paramagnetic particle is iron oxide having an associated electronic charge.

A method of the invention may also comprise a method as described above wherein said associated electronic charge is an overall positive charge.

A method of the invention may also comprise a method as described above wherein the attachment of ligands is used to associate said second electronic charge with said paramagnetic particle.

A method of the invention may also comprise a method as described above wherein said one or more proteins are modified to carry an overall negative charge.

A method of the invention may also comprise a method as described above wherein said modification of said one or more proteins comprises a modification selected from the group consisting of citraconylation, maleylation, trifluoroacetylation, tetraflurosuccinylation, succinylation and combinations thereof.

A method of the invention may also comprise a method as described above wherein said modification comprises the addition of a detergent.

A method of the invention may also comprise a method as described above wherein said detergent is sodium dodecylsulfate (SDS).

A method of the invention may also comprise a method as described above wherein said modification of said one or more proteins comprises modifying at least one lysine amino acid on said one or more proteins.

A method of the invention may also comprise a method as described above wherein said modification of said one or more proteins comprises modifying at least one arginine amino acid on said one or more proteins.

A method of the invention may also comprise a method as described above wherein said modification of said arginine amino acids comprises 1,2-cyclohexanedione.

A method of the invention may also comprise a method as described above wherein said one or more proteins are modified to carry an overall positive charge.

According to the invention, a method for extracting a protein of interest from sample may comprise: a) adding at least one paramagnetic particle to said sample; b) contacting said at least one paramagnetic particle with said sample to form a particle-protein complex between said protein of interest and said at least one paramagnetic particle; c) immobilizing said complex by applying a first magnetic field; d) removing material from said sample that is not immobilized by said first magnetic field; e) removing said first magnetic field from the remaining material to release said immobilized complex; f) disassociating said complex to create an extract solution comprising said protein of interest and said paramagnetic particles; and g) separating said paramagnetic particle from said extract solution, said separated extract solution comprising said protein of interest.

A method for fractionating a sample containing one or more proteins of interest and one or more proteins not of interest, according to a further aspect of the invention may comprise: a) adding at least one paramagnetic particle having a first electronic charge to said sample such that a particle-protein complex is formed between said at least one paramagnetic particle and said one or more proteins not of interest, said one or more proteins not of interest having a second electronic charge opposite to said at least one paramagnetic particle; b) immobilizing said complex by applying a magnetic field; and c) separating the sample portion not immobilized by said magnetic field from said immobilized complex, said separated sample portion containing said one or more proteins of interest.

### DETAILED DESCRIPTION OF THE INVENTION

The present invention relates to unique compositions of matter and their methods of use to extract proteins from crude biological sample solutions. The invention uses an electronically charged paramagnetic particle to bind proteins having a charge opposite that of the paramagnetic particle. The invention can be used to remove protein from a sample prior to releasing nucleic acid from a host cell or microorganism. The technique is helpful when a nucleic acid preparation free of protein is required. Likewise, the invention can be used to extract a subset of the total protein sample population by manipulating protein binding conditions. Using the invention for these purposes gives rise to two separate uses: (1) binding the protein of interest, discarding the unbound sample that may contain proteins not of interest, and eluting the bound proteins for further analysis; or (2) removing proteins not of interest from a sample containing a protein of interest, which may be subsequently separated for further analysis.

When the paramagnetic particles carry a charge, for example an electrical charge, these charged particles can reversibly bind to protein molecules having an overall charge opposite to that of the paramagnetic particle. The particle and the protein, therefore, bond to form a protein and particle complex.

Charge may be associated with the paramagnetic particle in any number of ways, and the invention is not be limited by the method of associating a charge with the particle. For example, a charge can be associated to the paramagnetic particle by attaching charged ligands to the paramagnetic particle. Ligands may include, but are not limited to, antibodies, haptens and receptors. In another embodiment, a charge can be associated to the paramagnetic particle by manipulating the pH, i.e., increasing or decreasing the pH, or ionic strength of the environment surrounding the particle. In either example, the overall charge on the paramagnetic particle can be positive or negative, depending on the ligand (anionic or cationic) or the pH of the solution environment.

Although not desiring to be bound by a particular theory, it is believed that when acid is used to associate charge, the acidic environment increases the electropositive nature of the metallic portion of the ferromagnetic particle. It is also believed that the low pH conditions increase the binding of the particles to the electronegative portions of a target compound, e.g., in proteins or polypeptides, or regions high in glutamic acid and aspartic acid.

As used herein, the term “paramagnetic particles” means particles capable of having a magnetic moment imparted to them when placed in a magnetic field. Typically, the particles consist of either metallic iron, cobalt or nickel, which are the only known elements that exist in a paramagnetic state while in their ground or zero oxidation state. In addition to these three metals, organic and organometallic compounds may also possess paramagnetic properties and may thus also be used. Paramagnetic particles, when placed in a magnetic field are movable under the action of the field. Such movement is useful for moving bound protein molecules in a sample processing protocol or other manipulations. Thus, protein molecules bound to the paramagnetic particles can be immobilized to the interior of a receptacle holding the protein sample or moved to different areas for exposure to different reagents and/or conditions with minimal direct contact.

The paramagnetic particles useful in the present invention need not be complicated structures. Suitable paramagnetic particles include, but are not limited to, iron particles, and the iron may be an iron oxide of forms such as, but not limited to, ferric hydroxide and ferrosoferric oxide, which have low solubility in an aqueous environment. Other iron particles such as iron sulfide and iron chloride may also be suitable for binding and extracting proteins using the conditions described herein.

Similarly, the shape of the paramagnetic particles is not critical to the present invention. The paramagnetic particles may be of various shapes including, for example, spheres, cubes, oval, capsule-shaped, tablet-shaped, nondescript random shapes, etc., and may be of uniform shape or non-uniform shapes. Whatever the shape of the ferromagnetic particles, the diameter at the widest point is generally in the range of from about 0.05 μm to about 50 μm, particularly from about 0.1 to about 0.3 μm.

In instances when acid or ionic strength is used to associate charge to the ferromagnetic particles or the target compounds, the pH or ionic strength can be provided through a variety of means. For example, the ferromagnetic particles can be added to an acidic solution or an acidic solution may be added to the particles. Alternatively, a solution or environment in which the ferromagnetic particles are located can be acidified by addition of an acidifying agent such as hydrochloric acid, sulfuric acid, phosphoric acid, acetic acid, citric acid or the like.

Provided that the environment in which the ferromagnetic particles are located is of a pH less than about 7.0, the particles will reversibly bind target molecules having an overall negative charge. Furthermore, the protein binding capacity of the ferromagnetic particles (without ligands or functional groups attached) increases as the pH decreases. Alternatively, as the solution approaches a neutral or higher pH, and the overall charge on the ferromagnetic particles become negative, positively-charged proteins can be bound. As shown below in Example 1, optimal extraction for the ferromagnetic particle, ferrosoferric oxide, occurs at pH ranges between 3-4 and 9-10.

As stated above, in an acidic environment, electropositive paramagnetic particles, such as ferric oxide particles, will bind electronegative protein molecules. Thus, the methods described herein can be used to fractionate proteins based on charge. In one embodiment of the present invention, reagents can be added to samples to impart overall negative charge on sample proteins, which can then bind electropositive paramagnetic particles. For example, lysine residues could be reversibly modified by citraconylation. Likewise, arginine residues could be modified by 1,2-cyclohexanedione. Other means of introducing a negative charge to proteins include maleylation, trifluoroacetylation, succinylation and tetrafluorosuccinylation. Various detergents, such as sodium dodecylsulfate (SDS), could also be used.

In another embodiment, protein modification can also be used to impart an overall positive charge on proteins, thereby preventing binding. This protein modification could be done to improve extraction efficiency and product purity by adding another means to fractionate the protein sample. Materials other than the protein to be bound therefore could be positively charged so that they are not attracted to the negatively charged paramagnetic reagent. The positively charged material would remain in solution so that it could be extracted from the bound protein held by the paramagnetic adduct. Such separation can be accomplished by means known to those skilled in the art such as centrifugation, filtering or application of magnetic force.

Once the protein molecules are bound, they can then be eluted into an appropriate buffer for further manipulation or characterization by various analytical techniques. The elution may be accomplished by heating and/or raising the pH. Agents that can be used to elute the protein from paramagnetic particles include, but are not limited to, basic solutions such as potassium hydroxide, sodium hydroxide or any compound that will increase the pH of the environment such that an electronegative protein will be displaced from the particles.

The following Example illustrates a specific embodiment of the invention described in this document. As would be apparent to skilled artisans, various changes and modifications are possible and are contemplated within the scope of the invention described.

### EXAMPLES

### Example 1

### Extraction of Protein From Human Plasma Samples Using Ferrosoferric Oxide

This example was performed to determine if ferrosoferic oxide particles at various pHs could be used to extract protein from human plasma samples, using an automated platform.

The materials used in this example were as follows:

(1) Human plasma samples

(2) 500 mM sample buffers
(a) Phosphoric Acid, pH 2;
(b) Citric Acid, pH 3;
(c) Citric Acid, pH 4;
(d) Citric Acid, pH 5;
(e) Citric Acid, pH 6;
(f) Phosphate, pH 7;
(g) Bicine, pH 8;
(h) Bicine, pH 9;
(i) Caps, pH 10; or
(j) Caps, pH 11

(3) Ferrosoferric Oxide

(4) BD Viper equipped with extraction block

(5) Baker Test strips

Each of the ten buffer solutions was mixed 1:1 with human plasma. The ten buffer solutions were also mixed 1:1 with distilled water. An aliquot (800 μl ) of each of the ten buffer:plasma and ten buffer:water samples was placed into an extraction tube, with each tube containing 100 mg of ferrosoferric oxide. Binding of protein to ferrosoferric oxide depended on the pH of the solution. The tubes were subsequently loaded into a BD Viper™ extraction block (Becton, Dickinson and Company). Each tube was subjected to forty-five (45) automated aspiration mixes to homogenize the mixtures, thereby further facilitating the complexing of the plasma protein and the ferrosoferric oxide. The protein/ferrosoferric oxide complex was then immobilized to the inside walls of extraction tubes using magnets that are integral to the BD Viper™ extraction block. Samples (200 μl) were taken from each of the extraction solutions and placed into empty wells of a multi-well collection device. The processed extraction solutions were diluted 1:25 in 500 mM KPO4 buffer to enable accurate absorbance analysis using spectroscopy at 280 nm.

TABLE I
Percentage Protein Recovery with Various Buffers

| | | | % Protein | |
|---|---|---|---|---|
| Sample Buffer | pH | % Protein Recovered | Extracted | Free Iron¹ |
| Phosphoric Acid | 2 | 136.5 | — | 5 mg/L |
| Citric Acid | 3 | 99.5 | 0.5 | 2-5 mg/L |
| Citric Acid | 4 | 90.9 | 9.1 | 2-5 mg/L |
| Citric Acid | 5 | 92.6 | 7.4 | — |
| Citric Acid | 6 | 99.8 | 0.2 | 0-2 mg/L |
| Phosphate | 7 | 98.6 | 1.4 | — |
| Bicine | 8 | 99.4 | 0.6 | — |
| Bicine | 9 | 97.3 | 2.7 | — |
| Caps | 10 | 96.3 | 3.7 | — |
| Caps | 11 | 99.2 | 0.8 | 0 mg/L |

¹Free iron in the extracts was characterized using Baker test strips on samples following extraction at pH 2, 3, 4, 6, and 11.

In buffers having a pH of 3 or less, minimal protein extraction was observed. Extraction was optimal at a pH range of 4-5 and, to a lesser extent, extraction was observed at a pH range of 9-10. A marked decrease in protein extraction was noted at more neutral pH ranges, (e.g., from 6-8) and under more basic conditions (e.g., at pH ranges 11 and above).

While the invention has been described with some specificity, modifications apparent to those with ordinary skill in the art may be made without departing from the scope of the invention. Various features of the invention are set forth in the following claims.

We claim:

1. A method for extracting protein from a sample comprising:
a) adding at least one paramagnetic particle comprising: a metal selected from the group consisting of iron, nickel and cobalt; a metal compound selected from the group consisting of iron oxide, iron sulfide, iron chloride, ferric hydroxide, ferrosoferric oxide, a cobalt compound, and a nickel compound; or an organometallic compound, to the sample comprising one or more proteins, at least one of said proteins having a first electronic charge;
b) associating a second electronic charge with the at least one paramagnetic particle, wherein the second electronic charge is opposite that of the first electronic charge such that the at least one paramagnetic particle and the proteins are capable of forming a protein-particle complex;
c) immobilizing the complex by applying a first magnetic field;
d) removing material from the sample that is not immobilized by the first magnetic field;
e) removing the first magnetic field from the remaining material to release the immobilized complex;
f) altering the second electronic charge on said at least one paramagnetic particle, such that the complex disassociates;
g) applying a second magnetic field to immobilize the at least one paramagnetic particle; and
h) extracting said protein from remaining material.

2. The method of claim 1, further comprising using an acid to associate said second electronic charge with the paramagnetic particle.

3. The method of claim 1, wherein the paramagnetic particle is iron oxide having an associated electronic charge.

4. The method of claim 1, wherein the associated second electronic charge is an overall positive charge.

5. The method of claim 1, further comprising using the attachment of ligands to associate the second electronic charge with the paramagnetic particle.

6. The method of claim 1, further comprising modifying the one or more proteins to carry an overall negative charge.

7. The method of claim 6, wherein the modification of the one or more proteins comprises a modification selected from the group consisting of citraconylation, maleylation, trifluoroacetylation, tetraflurosuccinylation, succinylation and combinations thereof.

8. The method of claim 6, wherein the modification of the one or more proteins comprises the addition of a detergent.

9. The method of claim 8, wherein the detergent is sodium dodecylsulfate (SDS).

10. The method of claim 6, wherein the modification of the one or more proteins comprises modifying at least one lysine amino acid on the one or more proteins.

11. The method of claim 6, wherein the modification of the one or more proteins comprises modifying at least one arginine amino acid on the one or more proteins.

12. The method of claim 11, wherein the modification of the arginine amino acids comprises 1,2-cyclohexanedione.

13. The method of claim 1, wherein the one or more proteins are modified to carry an overall positive charge.

14. A method for extracting a protein of interest from sample comprising:
a) adding at least one paramagnetic particle to the sample;
b) contacting the at least one paramagnetic particle with the sample to form a particle-protein complex between the protein of interest and the at least one paramagnetic particle;
c) immobilizing the complex by applying a first magnetic field;
d) removing material from the sample that is not immobilized by the first magnetic field;
e) removing the first magnetic field from the remaining material to release the immobilized complex;
f) disassociating the complex to create an extract solution comprising the protein of interest and the paramagnetic particles; and
g) separating the paramagnetic particle from the extract solution, the separated extract solution comprising the protein of interest.

15. A method for fractionating a sample containing one or more proteins of interest and one or more proteins not of interest, the method comprising:
a) adding at least one paramagnetic particle having a first electronic charge to the sample such that a particle-protein complex is formed between the at least one paramagnetic particle and the one or more proteins not of interest, the one or more proteins not of interest having a second electronic charge opposite to the at least one paramagnetic particle;
b) immobilizing the complex by applying a magnetic field; and
c) separating the sample portion not immobilized by the magnetic field from the immobilized complex, the separated sample portion containing the one or more proteins of interest
